(19) United States
(12) Patent Application Publication
(10) Pub. No.: US 2003/0039854 A1
(43) Pub. Date: Feb. 27, 2003

(54) Copolymeric binder for paperboard coatings with improved glueability and associated methods

(76) Inventors: Michael J. Drewery, Charlotte, NC (US); Ralph Lewis De Jong, Oakville (CA)

Correspondence Address: BASF CORPORATION, ANNE GERRY SABOURIN, 26701 TELEGRAPH ROAD, SOUTHFIELD, MI, 48034-2442, US

(21) Appl. No.: 09/930,586

(22) Filed: Aug. 15, 2001

(51) Int. Cl. A61L 2/00

(52) U.S. Cl. 428/511; 523/103; 428/518; 428/517

(57) ABSTRACT

Copolymeric binders for use in paperboard-coating compositions are provided, which exhibit lower gel content than the latices employed in conventional paperboard-coating compositions. Paperboard-coating compositions incorporating the copolymeric binders of the present invention exhibit improved glueability. More specifically, paperboard coated with the composition of the present invention requires less time to develop backside fiber tear in comparison to conventional paperboard, at no sacrifice to the remaining paperboard properties.

### FIELD OF THE INVENTION

The present invention relates to copolymeric binders for use in paperboard-coating compositions. More specifically, the present invention relates to copolymeric binders that provide improved glueability properties to paperboards coated therewith. In particular, paperboard coated with the copolymeric binders of the present invention exhibit improved time to develop backside fiber tear.

### BACKGROUND OF THE INVENTION

Paperboard is widely used in a variety of packaging applications. For example, paperboard is commonly employed to form cartons and cases, particularly printed cartons and cases containing glued seams. Depending on the particular end-use, the paperboard employed in packaging applications can be subject to numerous demanding specifications. Consequently, paperboard intended for use in packaging must advantageously exhibit a range of physical characteristics. In particular, printability and glueability are two of the primary physical characteristics required in paperboard for packaging applications.

Considerable strides have been taken in improving paperboard printability. For example, paperboard that is to be printed upon is ordinarily coated with a composition that includes a pigment and latex binder to provide a smooth surface upon which a glossy, brilliant, detailed inked image is applied. Two of the primary issues in improving printability are increasing the binding and tensile strengths of the paperboard coating. Binding strength is generally defined as the ability of the paperboard coating to withstand the printing process. Coatings possessing insufficient binding strength may pick or flake off during printing, thus giving rise to print defects. Tensile strength generally refers to the coating's ability to maintain its integrity under stress in a two-dimensional plane.

In addition to binding strength and tensile strength, coatings used in printed paperboard applications must also provide aesthetic characteristics, such as a suitable level of print gloss. It has heretofore been believed that high levels of gel content within the latex binder are required to provide adequate binding properties, tensile strength, and print gloss, and hence improved printability. In fact, the typical gel content for binders used in commercial paperboard coatings is in excess of 80%.

However, in addition to superior printability characteristics, paperboard must also provide acceptable gluing properties. Although great strides have been made in the area of improved printability, glueability continues to provide challenges within the paperboard industry. Further, the conditions employed during bonding can be quite demanding. For example, package users continually require products capable of processing at higher and higher speeds, translating into shorter glue setting times.

One particularly important aspect of glueability is the ability to quickly develop an acceptable level of bonding. Generally, acceptable bonding occurs when cohesive failure within the paperboard substrate itself arises, rather than adhesive failure between the layers, such as either between the substrate and the coating or the coating and the adhesive, or cohesive failure within either the adhesive or the coating. Several factors are involved in the rate at which acceptable bonding occurs. Coating strength also plays a fundamental role in glueability, including bonding speed. More specifically, if the coating is too weak, cohesive failure with the coating will be induced during testing, and fiber tear will not be achieved.

Another important factor in improving glueability is the dehydration rate, or the rate at which water or other solvent is transported from the adhesive. The speed of glueability is generally determined by measuring the time to develop adequate adhesion, i.e., sufficient adhesion to induce fiber tear, within a laminate formed from strips of paperboard.

To add to the complexity, the modifications made to satisfy the continual demand for improved printability typically decreases the resulting glueability performance of the paperboard, further exacerbating the delicate balance required in the production of paperboard. Coating compositions having high binding and tensile strengths may possess superior printability while exhibiting inferior glueability, for example. Further, in addition to providing adequate properties to the coated paperboard, the paperboard-coating composition must also possess suitable storage and processing characteristics. For example, the paperboard-coating composition may be required to provide a shelf life of up to two years. Further, the coating composition must not run, or weep, during application or buildup on the coating equipment over time. Accordingly, a myriad of factors must be balanced to provide paperboard-coating compositions suitable for commercial use.

### SUMMARY OF THE INVENTION

The present invention provides paperboard-coating compositions that impart improved glueability properties to coated paperboard substrates without sacrificing the remaining paperboard properties. More specifically, the present invention provides a coated paperboard that exhibits improved time to develop backside fiber tear without detriment to printability. Coated paperboard exhibiting greater than 15%; greater than 30%; and even up to a 45% decrease or more in time to obtain backside fiber tear can be produced from the compositions of the present invention, for example. This increase in glueability is further made without detriment to printability.

The paperboard-coating compositions of the present invention include a copolymeric binder that contains at least one alkenyl aromatic monomer, present in an amount ranging from about 30 to about 70 weight percent, based on total monomer content; at least one aliphatic conjugated diene monomer, present in an amount ranging from about 30 to about 70 weight percent, based on total monomer content; at least one unsaturated mono-carboxylic acid monomer, present in an amount ranging from about 1 to about 5 weight percent, based on total monomer content; and at least one unsaturated poly-carboxylic acid monomer, present in an amount ranging from about 1 to about 5 weight percent, based on total monomer content. In advantageous embodiments, the copolymeric binder further includes at least one chain transfer agent, present in an amount of at least about 1.0 weight percent, based on total monomer content. In further beneficial aspects of the invention, the copolymeric binder can include at least one cyanovinyl monomer, present in an amount of up to about 25 weight percent, based on total monomer content. The copolymeric binders of the present invention advantageously exhibit a solids content that ranges in amount from about 45 to about 55 weight percent, based on the weight of the binder. The copolymeric binders of the present invention beneficially exhibit a gel content ranging from about 45 to about 75%. Paperboard coated with a sufficient amount of the paperboard-coating composition of the present invention exhibits an improvement ranging from at least about 15 up to about 45% in time to obtain backside fiber tear.

A broad range of monomers are suitable for use within the copolymeric binder. For example, useful alkenyl aromatic monomers beneficially include styrene, α-methyl styrene, p-tertiary butyl styrene, methyl vinyl toluene, p-vinyl toluene, 3-ethyl styrene, or mixtures thereof. In further advantageous aspects, the alkenyl aromatic monomer is styrene. In beneficial embodiments of that aspect, the styrene may be present in amounts ranging from about 50 to about 57.5 weight percent, based on total monomer content. Suitable aliphatic conjugated dienes include 1,3-butadiene, 1,3-pentadiene, isoprene, 2,3-dimethyl-1,3-butadiene, or mixtures thereof. In beneficial aspects of the present invention, the aliphatic conjugated diene is 1,3-butadiene. In further embodiments of that aspect, the butadiene may be present in amounts ranging from about 38 to about 47 weight percent, based on total monomer content. Suitable unsaturated mono-carboxylic acid monomers include acrylic acid, methacrylic acid, crotonic acid, or mixtures thereof. Suitable unsaturated poly- or di-carboxylic acid monomers include maleic acid, fumaric acid, itaconic acid, and mixtures thereof. In advantageous aspects of the present invention, the mono-carboxylic acid is acrylic acid and the poly- or di-carboxylic acid is itaconic acid. In further embodiments, the acrylic acid can be present in amounts ranging from about 1.0 to about 4.0 weight percent, based on total monomer content, and the itaconic acid can be present in amounts ranging from about 0.5 to about 1.5 weight percent, based on total monomer content. Exemplary cyanovinyl monomers include acrylonitrile, methacrylonitrile, α-chloroacrylonitrile, and mixtures thereof, particularly acrylonitrile.

Chain transfer agents that can beneficially be employed include hexylmercaptan, octylmercaptan, n-dodecylmercaptan, tertiary dodecylmercaptan, n-hexadecylmercaptan, tert-hexadecylmercaptan, n-tetradecylmercaptan, tert-tetradecylmercaptan, dimethylxanthogen disulfide, diethylxanthogen disulfide, diisopropylxanthogen disulfide, tetramethylthiuram disulfide, tetraethylthiuram disulfide, tetrabutylthiuram disulfide, carbon tetrachloride, carbon tetrabromide, ethylene bromide, 2-ethylhexyl thioglycolate, α-methylstyrene dimer, terpinolene, α-terpinene, γ-terpinene, dipentene, and mixtures thereof. In advantageous embodiments, the chain transfer agent is tertiary dodecylmercaptan, particularly tertiary dodecylmercaptan present in amounts ranging from about 1.0 to about 1.5 weight percent, based on total monomer content.

Exemplary advantageous copolymeric binders for use in the paperboard-coating compositions of the present invention include copolymeric binders derived from monomer compositions that include styrene monomer, present in an amount ranging from about 50 to about 57.5 weight percent based on total monomer content; butadiene monomer, present in an amount ranging from about 38 to about 47 weight percent based on total monomer content; acrylic acid monomer, present in an amount ranging from about 1 to 4 weight percent based on total monomer content; itaconic acid monomer, present in an amount ranging from about 0.5 to 1.5 weight percent based on total monomer content; acrylonitrile monomer, present in an amount ranging from about 10 to about 25 weight percent based on total monomer content; and tertiary dodecylmercaptan, present in an amount ranging from about 1.0 to about 1.5 weight percent based on total monomer content. Copolymeric binders formed from such monomer compositions advantageously exhibit gel contents ranging from about 45 to about 75%.

The present invention further provides paperboard coatings incorporating the copolymeric binders described above. In addition to copolymeric binder, the paperboard-coating compositions can further include clay, mineral pigments, synthetic pigments, dispersants, thickeners, lubricants, insolubilizers, and the like.

Processes for preparing copolymeric binders, paperboard-coating compositions and coated paperboards in accordance with the present invention are also provided. Methods to prepare paperboard coatings generally include (a) preparing a copolymeric binder exhibiting a gel content ranging from about 45 to about 75% by polymerizing a monomeric composition that includes at least one alkenyl aromatic monomer; at least one aliphatic conjugated diene monomer; at least one unsaturated mono-carboxylic acid monomer; at least one unsaturated poly-carboxylic acid monomer; and, optionally, at least one cyanovinyl monomer and at least one chain transfer agent; (b) dispersing the copolymeric binder produced in step (a) in an aqueous medium; (c) incorporating at least one thickener and at least one pigment; (d) adding sufficient water to form about a 45 to about 70% solids coating composition; and (e) increasing the pH of the paperboard-coating composition. Suitable monomers and chain transfer agents are described above, as well as the amounts of the various components.

The present invention also provides paperboards coated with paperboard-coating compositions that typically include copolymeric binders derived from at least one alkenyl aromatic monomer, present in an amount ranging from about 30 to about 70 weight percent; at least one aliphatic conjugated diene monomer, present in an amount ranging from about 30 to about 70 weight percent; at least one unsaturated mono-carboxylic acid monomer, present in an amount ranging from about 1 to about 5 weight percent; at least one unsaturated poly-carboxylic acid monomer, present in an amount ranging from about 1 to about 5 weight percent; at least one cyanovinyl monomer, present in an amount of up to about 25 weight percent; and at least one chain transfer agent, present in an amount of at least about 1.0 weight percent, the copolymeric binder exhibiting a gel content ranging from about 45 to about 75%. The paperboard-coating composition optionally includes at least one thickener and at least one pigment. Advantageous aspects of the present invention provide methods by which to prepare such coated paperboard by applying the above-described paperboard-coating composition and drying the coated paperboard. Paperboard produced in accordance with the present invention exhibits a time to obtain backside fiber tear of less than 75 seconds, as measured using the Waldorf Glueability Test. The paperboard may further be coated with the paperboard-coating composition in an amount ranging from about 12 to about 25 parts of binder per 100 parts of pigment.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

The paperboard-coating compositions of the present invention are generally comprised of an aqueous dispersion containing a copolymeric binder derived from a monomer composition that includes at least one main monomer and at least one functional monomer. The main monomers include at least one alkenyl aromatic monomer and at least one aliphatic conjugated diene monomer. In addition, at least one cyanovinyl monomer may optionally be included as a main monomer. The functional monomers include at least one ethylenically unsaturated mono-carboxylic acid monomer and at least one ethylenically unsaturated poly-carboxylic acid monomer. The copolymeric binder generally further includes at least one chain transfer agent. In addition to the copolymeric binder, the paperboard-coating composition may advantageously contain a number of other components, such as dispersants, pigments, and the like.

The alkenyl aromatic monomer selected as a main monomer generally contributes to the binding strength and tensile strength of the paperboard-coating composition. Suitable alkenyl aromatic monomers include alkenyl aromatic compounds having from about 8 to about 12 total carbon atoms. Examples of specific alkenyl aromatic compounds include styrene, α-methyl styrene, p-tertiary butyl styrene, methyl vinyl toluene, p-vinyl toluene, 3-ethyl styrene, and mixtures thereof. In one particularly advantageous embodiment, styrene is employed. The alkenyl aromatic monomer may be present in the composition in amounts ranging from about 30 to about 70 weight percent, based on total monomer content. In further advantageous embodiments, the alkenyl aromatic monomer is present in amounts ranging from about 50 to about 57.5 weight percent, based on total monomer content. Copolymeric binders that include an insufficient amount of alkenyl aromatic monomer do not provide adequate binding and tensile properties. Conversely, copolymeric binders that include excessive amounts of alkenyl aromatic monomer exhibit inferior glueability performance.

Aliphatic conjugated diene monomers suitable for use in the present invention generally contain from about 4 to about 8 carbon atoms, desirably from about 4 to about 6 carbon atoms. Exemplary aliphatic conjugated diene monomers include 1,3-butadiene, piperylene, isoprene, 2,3-dimethyl-1,3-butadiene, and mixtures thereof. In one advantageous embodiment, butadiene is employed. The aliphatic conjugated diene monomers may be in an amount of about 30 to about 70% by weight, based on total monomer content. In advantageous embodiments, the conjugated diene monomers are present in amounts ranging from about 38.7 to about 47% by weight, based on total monomer content.

The aliphatic conjugated diene monomer also contributes to the binding and tensile strengths of the paperboard-coating composition. More particularly, a proper ratio of alkenyl aromatic monomer to aliphatic conjugated diene monomer provides paperboard-coating compositions that exhibit acceptable glass transition temperatures. Elevated glass transition temperatures are believed to be deleterious to coating strength. The glass transition temperature of the copolymeric binders of the present invention typically ranges from about −20 to 0° C.

Optionally, a cyanovinyl monomer is also incorporated as a main monomer in the copolymeric binder coating composition. Although not wishing to be bound by a particular theory, it is believed that the cyanovinyl monomer generally provides improved coating strengths and high ink gloss or print gloss values to the paperboard-coating compositions of the present invention. Any cyanovinyl monomer suitable for use in emulsion polymerization may be employed. Exemplary cyanovinyl monomers for use in the present invention include acrylonitrile, methacrylonitrile, α-chloroacrylonitrile, and mixtures thereof. In one advantageous embodiment, acrylonitrile is employed. The cyanovinyl monomer can be present in the copolymeric binder in an amount ranging from about 10 to about 25 weight percent, based on the total monomer content. In advantageous embodiments, the cyanovinyl monomer is present in an amount of about 15 weight percent, based on total monomer content.

The functional monomers used in the invention generally include a mixture of mono- and poly-carboxylic acids. Acrylamide may optionally be included as a functional monomer, as well. Applicants have determined that a mixture of ethylenically unsaturated mono-carboxylic and poly-carboxylic acids within the copolymeric binder provides improved shelf stability to the resulting paperboard binder. Shelf lives ranging from about 6 months up to about 2 years are possible with the copolymeric binder of the present invention. The stability imparted by the mixed carboxylic acids of the present invention is particularly beneficial in conjunction with the speeds employed in paperboard coating. The inclusion of insufficient amounts of carboxylic acids has also been found to detrimentally impact coating strengths. As a further benefit, the mixed carboxylic acids have been shown to produce less buildup within the coater over time. More specifically, the paperboard-coating compositions of the present invention exhibit superior runnability at speeds of about 1285 meters/min without severe buildup over time.

Any ethylenically unsaturated mono-carboxylic acid monomer suitable for use in emulsion polymerization can be employed in the present invention. Exemplary ethylenically unsaturated mono-carboxylic acid monomers include acrylic acid, methacrylic acid, crotonic acid, and mixtures thereof. In one beneficial embodiment, the ethylenically unsaturated mono-carboxylic acid is acrylic acid. The ethylenically unsaturated mono-carboxylic acid monomer can be present in amounts ranging from about 1 to about 10 weight percent, based on total monomer content. In advantageous embodiments, the ethylenically unsaturated mono-carboxylic acid monomer is present in amounts ranging from about 2.0 to about 2.5 weight percent, based on total monomer content.

Similarly, any unsaturated poly-carboxylic acid monomer suitable for use in emulsion polymerization may be employed in the present invention. As used herein, the term “poly-carboxylic acid” includes the respective anhydride thereof. In advantageous embodiments, dicarboxylic acid or dicarboxylic anhydride monomers are employed. Exemplary poly-carboxylic acid monomers include maleic acid, fumaric acid, itaconic acid, and mixtures thereof. The ethylenically unsaturated poly-carboxylic acid monomer can be present in an amount ranging from about 1 to about 10 weight percent, based on total monomer content. In advantageous embodiments, the ethylenically unsaturated multi-carboxylic acid monomer is present in amounts ranging from about 1.0 to about 1.25 weight percent, based on total monomer content.

Optionally, acrylamide can also be present as a functional monomer in amounts ranging from about 0.5 to about 2 weight percent, based on total monomer content.

The copolymeric binders of the present invention beneficially exhibit gel contents ranging from about 45 to about 75 percent. Although not wishing to be bound by a particular theory, Applicants believe that a gel content ranging from about 45 to about 75 percent contributes to the improved glueability properties of the present invention without detriment to the remaining properties. In particular, the copolymeric binders of the present invention provide improved time to develop backside fiber tear while surprisingly retaining acceptable printability. More specifically, Applicants believe that by employing the particular levels of styrene and gel content recommended in the present invention, the resulting viscoelastic properties of the copolymeric binder yield a significant improvement in time to develop fiber tear without detriment to the remainder of the coating properties. As noted above, the gel contents of the present invention are significantly lower than the gel contents provided in commercial paperboard coating formulations. In advantageous embodiments, the paperboard-coating compositions of the present invention provide improvements in time to develop fiber tear of greater than about 15%, more preferably, greater than about 30% (for example, from about 15 up to about 45%).

Several techniques can be used to reduce gel contents in accordance with the invention, including the use of chain transfer agents, stopping polymerization before complete conversion, lowering the polymerization temperature, as well as the use of specific initiator systems. One or more of these techniques may be employed in the present invention to lower gel content and preferably chain transfer agents are used to lower gel content.

Useful chain transfer agents, also commonly referred to as a molecular weight regulators, include any of those known chain transfer agents commonly used in emulsion polymerization. Hydrophobic chain transfer agents are particularly advantageous for use in the present invention. Exemplary hydrophobic chain transfer agents include alkylmercaptans containing at least 5 carbon atoms, such as tertiary dodecylmercaptan, hexylmercaptan, octylmercaptan, n-dodecylmercaptan, n-hexadecylmercaptan, tert-hexadecylmercaptan, n-tetradecylmercaptan; xanthogen disulfides, such as dimethylxanthogen disulfide, diethylxanthogen disulfide, and diisopropylxanthogen disulfide; thiuram disulfides, such as tetramethylthiuram disulfide, tetraethylthiuram disulfide and tetrabutylthiuram disulfide; halogenated hydrocarbons, such as carbon tetrachloride, carbon tetrabromide, and ethylene bromide; 2-ethylhexyl thioglycolate, α-methylstyrene dimer, terpinolene, α-terpinene, γ-terpinene, dipentene, and mixtures thereof. In advantageous embodiments, tertiary dodecylmercaptan (“TDM”) is employed as the chain transfer agent. The chain transfer agent can be present in the copolymeric binder in an amount ranging from about 1.0 to about 1.5 weight percent, based on total monomer content.

The copolymeric binders of the present invention are formed using emulsion polymerization. In general, the monomer compositions of the present invention are polymerized in the presence of water to form the copolymeric binder using conventional emulsion polymerization techniques, except as may be otherwise indicated. More specifically, the monomer compositions of the present invention are emulsion polymerized within a continuous aqueous phase which typically further includes one or more free radical initiators, emulsifiers, chelating agents, and the like.

Any free radical initiator suitable for use in emulsion polymerization may be utilized to polymerize the monomer composition, including sodium persulfate, ammonium persulfate, potassium persulfate, and the like. Other free radical initiators can be utilized which decompose or become active at the polymerization temperature such as various peroxides, e.g., cumene hydroperoxide, dibenzoyl peroxide, diacetyl peroxide, dodecanoyl peroxide, di-t-butyl peroxide, dilauroyl peroxide, bis(p-methoxy benzoyl) peroxide, t-butyl peroxy pivalate, dicumyl peroxide, isopropyl percarbonate, di-sec-butyl peroxidicarbonate, various azo initiators such as azobisdimethylvaleronitrile, 2,2′-azobisisobutyronitrile, 2,2′-azobis(2-amidinopropane) dihydrochloride, 2,2′-azobis-2-methylbutyronitrile, 2,2′-azobis (methylisobutyrate), and the like, and mixtures thereof The amount of the free radical initiator generally ranges from about 0.1 to 5 by weight percent, based on the weight of the monomer composition, as known in the art.

The emulsifier can generally be any surfactant, soap, or the like suitable for use in emulsion polymerization that is stable at the pH of the coating composition of the present invention. Examples of specific emulsifiers include alkyl sulfates, alkyl sulfosuccinates, alkyl aryl sulfonates, α-olefin sulfonates, fatty or rosin acid salts, nonyl or octyl phenol reaction products of ethylene oxide, and the like. The alkyl portion of the various emulsifiers generally has from about 8 to about 18 carbon atoms. Examples of specific surfactants include sodium lauryl sulfate, sodium sulfosuccinates such as sodium dimethylamyl sulfosuccinate, sodium dodecyl diphenylether disulfonate, and mixtures thereof. The amount of emulsifier present is sufficient to obtain an aqueous emulsion of the monomer composition, as known in the art. The emulsifiers are typically present in amounts ranging from about 0.5 to about 5 parts weight percent, based on the weight of the monomer composition.

Chelating agents may optionally be included in the monomer composition to tie up various metal impurities as well as to achieve a uniform polymerization. Examples of specific chelating agents include ethylene diamine tetra-acetic acid, nitrilotri-acetic acid, citric acid, and their ammonium, potassium, and sodium salts. Such chelating agents are typically present in amounts ranging from about 0.05 to about 0.5 weight percent, based on the weight of the monomer composition.

The pH of the emulsion polymerization typically ranges from about 2 to about 6. The pH is generally controlled by addition of a base, or more preferably a suitable buffer, or a mixture thereof, during the polymerization reaction. Examples of specific bases and buffers include ammonia, potassium hydroxide, sodium bicarbonate, and ammonium acetate, and the like. The amount of base or buffer added to the polymerization reaction is adjusted to obtain the desired pH range, as is well known in the art.

The polymerization process generally involves the addition polymerization of the main monomers and functional monomers. More particularly, the alkenyl aromatic monomer, aliphatic conjugated diene monomer, optional cyanovinyl monomer, unsaturated mono-carboxylic acid monomer, unsaturated poly-carboxylic acid monomer, and optional acrylamide monomer are subjected to emulsion polymerization. In advantageous aspects of the present invention, the monomer composition includes styrene, butadiene, acrylic acid, itaconic acid, and acrylonitrile. The various monomers may be added either continuously or in a batch process to a reaction zone of an emulsion polymerization reactor, as is known in the art.

The emulsion polymerization process typically begins by charging a reactor with water, an emulsifier, a sufficient amount of a buffer or base, and, optionally, the chain transfer agent. Subsequently, the main and functional monomers are introduced into the reactor. The order of the main and functional monomer addition is not critical to the reaction. Then, additional water and polymerization catalyst are added to the reactor and the monomer composition is allowed to react to completion.

The polymerization reaction is generally carried out at a temperature from about 165° F. to 210° F., and preferably at about 194° F. Polymerization is generally conducted for about 2.5 to 6 hours; however, polymerization conditions may vary as desired to provide different conversion levels of monomer composition to copolymeric binder. The monomer composition is typically allowed to react in the reactor until at least about 99 percent of the monomer has been converted. The copolymeric binder typically ranges in solids from about 45 to 55 percent following the polymerization reaction.

Paperboard-coating compositions incorporating the copolymeric binders of the present invention may generally further include one or more pigments, dispersants, thickeners, lubricants, insolubilizers, or any of the numerous paper coating additives well known to those skilled in the art, dispersed within an aqueous medium.

Examples of pigments include clay, such as kaolin clay, and mineral pigments, such as titanium dioxide, calcium carbonate, zinc oxide, and the like. Synthetic pigments, or plastic pigments, such as polystyrene, may also be beneficially employed. The particle sizes of the pigments generally vary from about 0.5 to 2 microns on the average. Suitable dispersants include sodium polyacrylates, such as DISPEX® N-40 dispersant, commercially available from Allied Colloids. Exemplary thickeners include STEROCOLL® FD thickener, a polyacrylate product commercially available from BASF Corporation. Suitable lubricants include calcium stearate, such as NOPCOTE® C104 lubricant, commercially available from Henkel Corporation. Exemplary insolubilizers include CURESAN® 199 insolubilizer, commercially available from BASF Corporation. Further, natural or synthetic co-binders may advantageously be employed. Natural co-binders include starches, and proteins such as casein and soy protein that are typically chemically-modified to make them suitable for paper coating compositions. Polyvinyl alcohol is an example of a synthetic co-binder that is sometimes used. Foam control agents and preservatives may also beneficially be employed.

Typically, the paperboard-coating composition includes 100 parts pigment, of which from about 0 to about 70 weight percent is clay and from about 0 to about 70 weight percent is calcium carbonate and from about 0 to about 10 weight percent is synthetic pigment. The coating composition further includes from about 12 to about 25 weight percent of the copolymeric binder of the present invention. The paperboard-coating compositions of the present invention typically range in percent solids from about 45 to about 70 percent by weight. The paperboard-coating composition is formed by dispersing the various components in an aqueous medium under sufficient agitation.

The coating compositions described herein may be applied to paperboard webs using any of the conventional coating devices such as blade coaters, air knife coaters, rod coaters, roll coaters and the like, by methods known to those skilled in the art. Exemplary coating weights which are applied range from about 5 to about 25 g/m². The paperboard-coating compositions of the present invention may further be applied to a range of paperboard substrates including virgin and recycled kraft, high and low density kraft, chipboard, and various types of treated and coated kraft and chipboard. A starch pre-coat may be employed to promote adhesion of the paperboard-coating composition to the substrate.

Paperboard coated with the compositions of the present invention provide a range of beneficial properties, including improved glueability at no sacrifice to printability. Coated paperboard formed in accordance with the present invention can exhibit up to a 45% improvement in time to obtain backside fiber tear. Further, the coated paperboards exhibit binding strengths ranging from about 120 to about 170 IGT dry pick based on #3 oil and 3 m/sec printing speed. The coated paperboards of the present invention also provide print gloss values of greater than 68.

The present invention will be illustrated in greater detail by the following specific examples. It is understood that these examples are given by way of illustration and are not meant to limit the disclosure of the claims to follow. All percentages in the examples, and elsewhere in the specification, are by weight unless otherwise specified.

### EXAMPLES

Unless noted to the contrary, the copolymeric binder emulsion polymerization reactions further described below were conducted in a one-gallon stainless steel reactor. The stainless steel reactor further included an agitator and a jacket capable of supplying either hot or cold water. Following the addition of the emulsifier, buffer or base, chain transfer agent, polymerization catalyst, and the main and functional monomer packages, the reactor was sealed, brought up to temperature, and allowed to react to completion. The progress of the polymerization reaction was monitored by measuring percent total solids. The polymerization reactions were considered complete when solids were about 98 to 100% of theory. After completion of the polymerization reaction, the copolymeric binder was discharged, cooled to room temperature, and the contents filtered. Steam stripping was used to further lower the volatile content of the dispersion.

All weight percents within the Examples are based on the total monomer content, unless noted to the contrary.

### Comparative Example 1

Dispersion A was prepared via the emulsion polymerization of 1700 g of a monomer mixture containing 50 weight percent styrene; 47 weight percent butadiene; 2.0 weight percent acrylic acid; and 1.0 weight percent itaconic acid. The gel content of Dispersion A was adjusted to 87 percent by including 0.5 weight percent of SULFOLE® 120 TDM (commercially available from Phillips Petroleum) within the reaction mixture. The gel content was determined by immersing a sample in toluene solvent for 48 hours.

The emulsion polymerization was conducted in a continuous phase comprising 1700 g deionized water and 14 g of AEROSOL MA® surfactant, commercially available from Henkel. The reaction temperature was 194° F. The percent solids of Dispersion A was 50%.

A 50 percent solids paperboard-coating composition incorporating Dispersion A was prepared by dispersing the following components in deionized water under constant agitation: 75 grams of No. 1 Standard Clay pigment, commercially available from Huber; 20 grams of titanium oxide pigment from DuPont; 5 grams of plastic pigment, commercially available as STYRONAL® BN 4901X pigment from BASF Corporation; and 18 grams of Dispersion A. Aqueous ammonia was then used to bring the pH of the paperboard-coating composition up to 9.0.

A coated paperboard sample was prepared by double coating a 2″×14″ strip of unbleached Kraft paperboard with the paperboard-coating composition incorporating Dispersion A using wire-wound rods. The resulting coatweight was 10 g/m². The paperboard was dried at 100° C. for 45 seconds following coating. The dried samples were conditioned under TAPPI standard conditions prior to testing.

### Comparative Example 2

Dispersion B was prepared using the procedures, materials, and amounts of Comparative Example 1, except that the monomer mixture contained 57.5 weight percent styrene; 38.75 weight percent butadiene; 2.5 weight percent acrylic acid; and 1.25 weight percent itaconic acid. The gel content of Dispersion B was adjusted to 85 percent by including 0.5 weight percent of TDM within the reaction mixture.

The same procedures, materials, and amounts used in Comparative Example 1 were employed to form a paperboard-coating composition incorporating Dispersion B in lieu of Dispersion A and respective coated paperboard samples therefrom.

### Example 1

Dispersion C was prepared using the procedures, materials, and amounts of Comparative Example 1, except that the monomer mixture contained 50.0 weight percent styrene; 47 weight percent butadiene; 2 weight percent acrylic acid; and 1 weight percent itaconic acid.

The gel content of Dispersion A was adjusted to 50 percent by including 1.5 weight percent TDM in the reaction mixture.

The same procedures, materials, and amounts used in Comparative Example 1 were employed to form a paperboard-coating composition incorporating Dispersion C in lieu of Dispersion A and respective coated paperboard samples therefrom.

### Example 2

Dispersion D was prepared using the procedures, materials, and amounts of Comparative Example 1, except that the monomer mixture contained 57.5 weight percent styrene; 38.75 weight percent butadiene; 2.5 weight percent acrylic acid; and 1.25 weight percent itaconic acid. The gel content of Dispersion D was adjusted to 50 percent by including 1.5 weight percent of TDM in the reaction mixture.

The same procedures, materials, and amounts used in Comparative Example 1 were employed to form a paperboard-coating composition incorporating Dispersion D in lieu of Dispersion A and respective coated paperboard samples therefrom.

### Example 3

Dispersion E was prepared using the procedures, materials, and amounts of Comparative Example 1, except that the monomer mixture contained 50.0 weight percent styrene; 46.3 weight percent butadiene; 2.5 weight percent acrylic acid; and 1.25 weight percent itaconic acid. The gel content of Dispersion E was adjusted to 75 percent by including 1.0 weight percent of TDM in the reaction mixture.

The same procedures, materials, and amounts used in Comparative Example 1 were employed to form a paperboard-coating composition incorporating Dispersion E in lieu of Dispersion A and respective coated paperboard samples therefrom.

### Testing of Comparative Examples 1 and 2 and Examples 1-3

The paperboard samples prepared in Comparative Examples 1 and 2 and Examples 1 to 3 were tested for glueability to determine time to obtain backside fiber tear. Both a manual pull test and the Waldorf Glueability Test were employed to measure the time to develop backside fiber tear.

In the manual pull test, commercial polyvinyl acetate glue (PVAc) is drawn down a strip of uncoated paperboard with a 3 mil. Bird bar. A 2″×14″ strip of coated paperboard is placed overtop the adhesive layer, and the bond between coated and uncoated paperboards is formed by rolling a 10 lb. wheel over the sample six times. After a one-minute delay, the top board is pulled up at a 90° angle in 1-2 inch increments every 15 seconds. The time to obtain fiber tear on the coated side is measured.

The WALDORF GLUEABILITY TESTERS performs a similar test using a continuous pull. A 2″×14″ laminate is formed by joining coated and uncoated surfaces of two paperboards using basic industry standard glue. The bond between coated and uncoated paperboards is formed with six passes of a wheel attachment. After a 45 second delay, the top board, which is the coated board, is continuously pulled at a 180° angle at a speed of 0.5 inches/sec. The time to obtain fiber tear on the coated side is measured.

The glueability results for Comparative Examples 1 and 2 and Examples 1-3 are provided in Table I below.

TABLE 1
Time to Induce
Backside Fiber Tear

| | Manual | Waldorf | |
|---|---|---|---|
| | Glueability Test | Glueability Test | Gel Content |
| | (sec) [Avg of 2] | (sec) [Avg of 2] | (%) |
| Comparative | 150 | 90 | 87 |
| Example 1 | | | |
| Comparative | 150 | 85 | 85 |
| Example 2 | | | |
| Example 1 | 83 | 69 | 50 |
| Example 2 | 105 | 70 | 50 |
| Example 3 | 105 | 71 | 75 |

From these examples, it can be seen that coatings made with copolymeric binders having gel levels in the preferred range of 45-75% in accordance with the invention have much faster times to develop backside fiber tear than dispersion binders with higher gel content. The paperboard coatings of the present invention develop backside fiber tear in well under 75 or 110 seconds, as measured using the Waldorf Glueability Test and Manual Glueability Test, respectively. Accordingly, as discussed previously, copolymeric binders characterized by lower gel contents exhibit improved glueability in comparison to conventional paperboard-coating compositions.

Further, the amounts of alkenyl aromatic monomer and chain transfer agents employed within the copolymeric binders of the present invention provide paperboard-coating compositions characterized by a balance of beneficial properties. For example, coated paperboards of the present invention exhibit binding strengths of at least about 120 and print gloss values of at least about 68. The copolymeric binders and paperboard-coating compositions of the invention further exhibit superior stability. In particular, the copolymeric binder compositions can provide shelf lives of up to about two years.

That which is claimed:

1. A copolymeric binder for use in paperboard-coating compositions, said copolymeric binder derived from monomers comprising:
(a) at least one alkenyl aromatic monomer, present in an amount ranging from about 30 to about 70 weight percent, based on total monomer content;
(b) at least one aliphatic conjugated diene monomer, present in an amount ranging from about 30 to about 70 weight percent, based on total monomer content;
(c) at least one unsaturated mono-carboxylic acid monomer, present in an amount ranging from about 1 to about 5 weight percent, based on total monomer content; and
(d) at least one unsaturated poly-carboxylic acid monomer, present in an amount ranging from about 0.5 to about 5 weight percent, based on total monomer content,
wherein said copolymeric binder exhibits a gel content ranging from about 45 to about 75%.

2. A copolymeric binder according to claim 1, wherein said copolymeric binder further comprises at least one chain transfer agent, present in an amount of at least about 1.0 weight percent, based on total monomer content.

3. A copolymeric binder according to claim 2, wherein said chain transfer agent is selected from the group consisting of hexylmercaptan, octylmercaptan, n-dodecylmercaptan, tertiary dodecylmercaptan, n-hexadecylmercaptan, tert-hexadecylmercaptan, n-tetradecylmercaptan, tert-tetradecylmercaptan, dimethylxanthogen disulfide, diethylxanthogen disulfide, diisopropylxanthogen disulfide, tetramethylthiuram disulfide, tetraethylthiuram disulfide, tetrabutylthiuram disulfide, carbon tetrachloride, carbon tetrabromide, ethylene bromide, 2-ethylhexyl thioglycolate, α-methylstyrene dimer, terpinolene, α-terpinene, γ-terpinene, dipentene, and mixtures thereof.

4. A copolymeric binder according to claim 2, wherein said chain transfer agent is tertiary dodecylmercaptan.

5. A copolymeric binder according to claim 2, wherein said chain transfer agent is present in an amount ranging from about 1.0 to about 1.5 weight percent.

6. A copolymeric binder according to claim 1, wherein said copolymeric binder further comprises at least one cyanovinyl monomer, present in an amount of up to about 25 weight percent, based on total monomer content.

7. A copolymeric binder according to claim 6, wherein said cyanovinyl monomer is selected from the group consisting of acrylonitrile, methacrylonitrile, α-chloroacrylonitrile, and mixtures thereof.

8. A copolymeric binder according to claim 6, wherein said cyanovinyl monomer is acrylonitrile.

9. A copolymeric binder according to claim 1, wherein said alkenyl aromatic monomer is selected from the group consisting of styrene, α-methyl styrene, p-tertiary butyl styrene, methyl vinyl toluene, p-vinyl toluene, 3-ethyl styrene, and mixtures thereof.

10. A copolymeric binder according to claim 1, wherein said alkenyl aromatic monomer is styrene.

11. A copolymeric binder according to claim 1, wherein said alkenyl aromatic monomer is present in an amount ranging from about 50 to about 57.5 weight percent.

12. A copolymeric binder according to claim 1, wherein said aliphatic conjugated diene is selected from the group consisting of 1,3-butadiene, 1,3-pentadiene, isoprene, 2,3-dimethyl-1,3-butadiene, and mixtures thereof.

13. A copolymeric binder according to claim 1, wherein said aliphatic conjugated diene is butadiene.

14. A copolymeric binder according to claim 1, wherein said aliphatic conjugated diene is present in an amount ranging from about 38 to about 47 weight percent.

15. A copolymeric binder according to claim 1, wherein said unsaturated mono-carboxylic acid monomer is selected from the group consisting of acrylic acid, methacrylic acid, crotonic acid, and mixtures thereof.

16. A copolymeric binder according to claim 1, wherein said unsaturated poly-carboxylic acid monomer is selected from the group consisting of maleic acid, fumaric acid, itaconic acid, and mixtures thereof.

17. A copolymeric binder according to claim 1, wherein said mono-carboxylic acid monomer is acrylic acid and said poly-carboxylic acid monomer is itaconic acid.

18. A copolymeric binder according to claim 1, wherein said unsaturated mono-carboxylic acid monomer is present in an amount ranging from about 2.0 to about 2.5 weight percent and said unsaturated poly-carboxylic acid monomer is present in an amount ranging from about 1.0 to about 1.25 weight percent.

19. A copolymeric binder according to claim 1, wherein said copolymeric binder further comprises acrylamide.

20. A copolymeric binder according to claim 19, wherein said acrylamide is present in an amount ranging from about 0.5 to about 2 weight percent, based on total monomer content.

21. A copolymeric binder according to claim 1, wherein said copolymeric binder exhibits a solids content which ranges from about 45 to about 55 weight percent, based on the weight of the binder.

22. A copolymeric binder for use in paperboard-coating compositions, said copolymeric binder derived from monomers comprising:
(a) at least one alkenyl aromatic monomer, present in an amount ranging from about 30 to about 70 weight percent, based on total monomer content;
(b) at least one aliphatic conjugated diene monomer, present in an amount ranging from about 30 to about 70 weight percent, based on total monomer content;
(c) at least one unsaturated mono-carboxylic acid monomer, present in an amount ranging from about 1 to about 5 weight percent, based on total monomer content;
(d) at least one unsaturated poly-carboxylic acid monomer, present in an amount ranging from about 1 to about 5 weight percent, based on total monomer content;
(e) at least one cyanovinyl monomer, present in an amount of up to about 25 weight percent, based on total monomer content; and
(f) at least one chain transfer agent, present in an amount of at least about 1.0 weight percent, based on total monomer content,
wherein said copolymeric binder exhibits a gel content ranging from about 45 to about 75%.

23. A copolymeric binder for use in paperboard-coating compositions, said copolymeric binder derived from monomers comprising:
(a) styrene monomer, present in an amount ranging from about 50 to about 57.5 weight percent, based on total monomer content;
(b) butadiene monomer, present in an amount ranging from about 38 to about 47 weight percent, based on total monomer content;
(c) acrylic acid monomer, present in an amount ranging from about 2.0 to about 2.5 weight percent, based on total monomer content;
(d) itaconic acid monomer, present in an amount ranging from about 1.0 to about 1.25 weight percent, based on total monomer content;
(e) acrylonitrile monomer, present in an amount ranging from about 10 to about 25 weight percent, based on total monomer content; and
(f) tertiary dodecylmercaptan, present in an amount ranging from about 1.0 to about 1.5 weight percent, based on total monomer content,
wherein said copolymeric binder exhibits a gel content ranging from about 45 to about 75%.

24. A paperboard-coating composition, comprising:
(a) a copolymeric binder, said copolymeric binder derived from monomers comprising:
(i) at least one alkenyl aromatic monomer, present in an amount ranging from about 30 to about 70 weight percent, based on total monomer content;
(ii) at least one aliphatic conjugated diene monomer, present in an amount ranging from about 30 to about 70 weight percent, based on total monomer content;
(iii) at least one unsaturated mono-carboxylic acid monomer, present in an amount ranging from about 1 to about 5 weight percent, based on total monomer content;
(iv) at least one unsaturated poly-carboxylic acid monomer, present in an amount ranging from about 1 to about 5 weight percent, based on total monomer content;
(v) at least one cyanovinyl monomer, present in an amount of up to about 25 weight percent, based on total monomer content; and
(b) water;
wherein said copolymeric binder exhibits a gel content ranging from about 45 to about 75% and solids ranging from about 45 to about 55%, said copolymeric binder further present in said paperboard-coating composition in an amount ranging from about 12 to about 25 weight percent, based on the weight of the paperboard-coating composition.

25. A paperboard-coating composition according to claim 24, wherein said paperboard-coating composition further comprises one or more pigments.

26. A paperboard-coating composition according to claim 25, wherein said pigments are selected from the group consisting of clay, mineral pigments, and synthetic pigments.

27. A paperboard-coating composition according to claim 24, wherein said paperboard-coating composition further comprises one or more components selected from the group consisting of dispersants, thickeners, lubricants, and insolubilizers.

28. A method for preparing a paperboard-coating composition comprising:
(a) preparing a copolymeric binder exhibiting a gel content ranging from about 45 to about 75% derived from monomers comprising:
(i) at least one alkenyl aromatic monomer, present in an amount ranging from about 30 to about 70 weight percent, based on total monomer content;
(ii) at least one aliphatic conjugated diene monomer, present in an amount ranging from about 30 to about 70 weight percent, based on total monomer content;
(iii) at least one unsaturated mono-carboxylic acid monomer, present in an amount ranging from about 1 to about 5 weight percent, based on total monomer content;
(iv) at least one unsaturated poly-carboxylic acid monomer, present in an amount ranging from about 1 to about 5 weight percent, based on total monomer content;
(v) optionally at least one cyanovinyl monomer, present in an amount of up to 25 weight percent, based on total monomer content; and
(vi) optionally at least one chain transfer agent, present in an amount of at least about 1.0 weight percent, based on total monomer content;
(b) dispersing said copolymeric binder in an aqueous medium;
(c) incorporating at least one thickener and at least one pigment;
(d) adding sufficient water to form an about 45 to about 70% solids coating composition; and
(e) increasing the pH of the paperboard-coating composition.

29. A method according to claim 28, wherein said alkenyl aromatic monomer is selected from the group consisting of styrene, α-methyl styrene, p-tertiary butyl styrene, methyl vinyl toluene, p-vinyl toluene, 3-ethyl styrene, and mixtures thereof.

30. A method according to claim 28, wherein said alkenyl aromatic monomer is styrene.

31. A method according to claim 28, wherein said alkenyl aromatic monomer is present in amounts ranging from about 50 to about 57.5 weight percent.

32. A method according to claim 28, wherein said aliphatic conjugated diene is selected from the group consisting of butadiene, 1,3-pentadiene, isoprene, 2,3-dimethyl-1,3-butadiene, and mixtures thereof.

33. A method according to claim 28, wherein said aliphatic conjugated diene is butadiene.

34. A method according to claim 28, wherein said aliphatic conjugated diene is present in amounts ranging from about 38 to about 47 weight percent.

35. A method according to claim 28, wherein said unsaturated mono-carboxylic acid monomer is selected from the group consisting of acrylic acid, methacrylic acid, crotonic acid, and mixtures thereof.

36. A method according to claim 28, wherein said unsaturated poly-carboxylic acid monomer is selected from the group consisting of itaconic acid, maleic acid, fumaric acid, and mixtures thereof.

37. A method according to claim 28, wherein said mono-carboxylic acid monomer is acrylic acid and said poly-carboxylic acid monomer is itaconic acid.

38. A method according to claim 28, wherein said mono-carboxylic acid monomer is present in amounts ranging from about 2.0 to about 2.5 weight percent and said poly-carboxylic acid monomer is present in amounts ranging from about 1.0 to about 1.25 weight percent.

39. A method according to claim 28, wherein said vinyl cyanide is acrylonitrile.

40. A method according to claim 28, wherein said chain transfer agent is selected from the group consisting of hexylmercaptan, octylmercaptan, n-dodecylmercaptan, tertiary dodecylmercaptan, n-hexadecylmercaptan, tert-hexadecylmercaptan, n-tetradecylmercaptan, tert-tetradecylmercaptan, dimethylxanthogen disulfide, diethylxanthogen disulfide, diisopropylxanthogen disulfide, tetramethylthiuram disulfide, tetraethylthiuram disulfide, tetrabutylthiuram disulfide, carbon tetrachloride, carbon tetrabromide, ethylene bromide, 2-ethylhexyl thioglycolate, α-methylstyrene dimer, terpinolene, α-terpinene, γ-terpinene, dipentene, and mixtures thereof.

41. A method according to claim 28, wherein said chain transfer agent is tertiary dodecylmercaptan.

42. A method according to claim 28, wherein said chain transfer agent is present in an amount ranging from about 1.0 to about 1.5 weight percent.

43. A method according to claim 28, wherein said pigment is selected from the group consisting of clay, mineral pigment, and synthetic pigment.

44. A method for preparing coated paperboard comprising:
(a) applying a paperboard-coating composition to at least one surface of the paperboard in an amount ranging from about 5 to about 25 g/m²; said paperboard coating comprising a copolymeric binder exhibiting a gel content ranging from about 45 to about 75%, said copolymeric binder derived from monomers comprising:
(i) at least one alkenyl aromatic monomer, present in an amount ranging from about 30 to about 70 weight percent, based on total monomer content;
(ii) at least one aliphatic conjugated diene monomer, present in an amount ranging from about 30 to about 70 weight percent, based on total monomer content;
(iii) at least one unsaturated mono-carboxylic acid monomer, present in an amount ranging from about 1 to about 5 weight percent, based on total monomer content;
(iv) at least one unsaturated poly-carboxylic acid monomer, present in an amount ranging from about 1 to about 5 weight percent, based on total monomer content;
(v) at least one cyanovinyl monomer, present in an amount of up to 15 weight percent, based on total monomer content; and
(vi) at least one chain transfer agent, present in an amount of at least about 1.0 weight percent based on total monomer content; and
(b) drying the coated paperboard,
wherein said dried paperboard exhibits a time to develop backside fiber tear of less than 75 seconds, as measured using the Waldorf Glueability Test.

45. A method according to claim 44, wherein said alkenyl aromatic monomer is selected from the group consisting of styrene, α-methyl styrene, p-tertiary butyl styrene, methyl vinyl toluene, p-vinyl toluene, 3-ethyl styrene, and mixtures thereof.

46. A method according to claim 44, wherein said alkenyl aromatic monomer is styrene.

47. A method according to claim 44, wherein said alkenyl aromatic monomer is present in amounts ranging from about 50 to about 57.5 weight percent.

48. A method according to claim 44, wherein said aliphatic conjugated diene is selected from the group consisting of butadiene, 1,3-pentadiene, isoprene, 2,3-dimethyl-1,3-butadiene, and mixtures thereof.

49. A method according to claim 44, wherein said aliphatic conjugated diene is butadiene.

50. A method according to claim 44, wherein said aliphatic conjugated diene is present in amounts ranging from about 38 to about 47 weight percent.

51. A method according to claim 44, wherein said unsaturated mono-carboxylic acid monomer is selected from the group consisting of acrylic acid, methacrylic acid, crotonic acid, and mixtures thereof.

52. A method according to claim 44, wherein said unsaturated poly-carboxylic acid monomer is selected from the group consisting of itaconic acid, maleic acid, fumaric acid, and mixtures thereof.

53. A method according to claim 44, wherein said mono-carboxylic acid monomer is acrylic acid and said poly-carboxylic acid monomer is itaconic acid.

54. A method according to claim 44, wherein said mono-carboxylic acid monomer is present in amounts ranging from about 2.0 to about 2.5 weight percent and said poly-carboxylic acid monomer is present in amounts ranging from about 1.0 to about 1.25 weight percent.

55. A method according to claim 44, wherein said vinyl cyanide is acrylonitrile.

56. A method according to claim 44, wherein said chain transfer agent is selected from the group consisting of hexylmercaptan, octylmercaptan, n-dodecyhnercaptan, tertiary dodecylmercaptan, n-hexadecylmercaptan, tert-hexadecylmercaptan, n-tetradecylmercaptan, tert-tetradecylmercaptan, dimethylxanthogen disulfide, diethylxanthogen disulfide, diisopropylxanthogen disulfide, tetramethylthiuram disulfide, tetraethylthiuram disulfide, tetrabutylthiuram disulfide, carbon tetrachloride, carbon tetrabromide, ethylene bromide, 2-ethylhexyl thioglycolate, α-methylstyrene dimer, terpinolene, α-terpinene, γ-terpinene, dipentene, and mixtures thereof.

57. A method according to claim 44, wherein said chain transfer agent is tertiary dodecylmercaptan.

58. A method according to claim 44, wherein said chain transfer agent is present in an amount ranging from about 1.0 to about 1.5 weight percent.

59. A method according to claim 44, wherein said applying step further comprises the steps of:
(a) incorporating at least one thickener and at least one pigment into the paperboard-coating composition;
(b) adding sufficient water to form an about 45 to 70% solids paperboard-coating composition; and
(c) increasing the pH of the paperboard-coating composition.

60. A method according to claim 44, wherein said pigment is selected from the group consisting of clay, mineral pigment, and synthetic pigment.

61. Paperboard coated with a coating composition comprising a copolymeric binder, said copolymeric binder derived from monomers comprising:
(a) at least one alkenyl aromatic monomer, present in an amount ranging from about 30 to about 70 weight percent, based on total monomer content;
(b) at least one aliphatic conjugated diene monomer, present in an amount ranging from about 30 to about 70 weight percent, based on total monomer content;
(c) at least one unsaturated mono-carboxylic acid monomer, present in an amount ranging from about 1 to about 5 weight percent, based on total monomer content;
(d) at least one unsaturated poly-carboxylic acid monomer, present in an amount ranging from about 1 to about 5 weight percent, based on total monomer content; and
(e) at least one cyanovinyl monomer, present in an amount of up to about 25 weight percent, based on total monomer content,
wherein said copolymeric binder exhibits a gel content ranging from about 45 to about 75%.

62. Paperboard according to claim 61, wherein said copolymeric binder further comprises at least one chain transfer agent, present in an amount of at least about 1.0 weight percent.

63. Paperboard according to claim 62, wherein:
(a) said alkenyl aromatic monomer is styrene monomer, present in an amount ranging from about 50 to about 57.5 weight percent;
(b) said aliphatic conjugated diene monomer is butadiene, present in an amount ranging from about 38 to about 47 weight percent;
(c) said unsaturated mono-carboxylic acid monomer is acrylic acid, present in an amount ranging from about 2.0 to about 2.5 weight percent;
(d) said unsaturated poly-carboxylic acid monomer is itaconic acid, present in an amount ranging from about 1.0 to about 1.25 weight percent; and
(e) said chain transfer agent is tertiary dodecylmercaptan, present in an amount ranging from about 1.0 to about 1.5 weight percent.

64. Paperboard coated with a composition according to claim 61, wherein said coating composition is present on the surface of the paperboard in coating weights ranging from about 5 to about 25 g/m².
